(19) United States
(12) Patent Application Publication
(10) Pub. No.: US 2006/0135461 A1
(43) Pub. Date: Jun. 22, 2006

(54) Reduction of hair growth

(76) Inventors: Natalia Botchkareva, Sharon, MA (US); Gurpreet S. Ahluwalia, Wellesley, MA (US)

Correspondence Address: FISH & RICHARDSON P.C., P.O. BOX 1022, MINNEAPOLIS, MN, 55440-1022, US

(21) Appl. No.: 11/313,501

(22) Filed: Dec. 21, 2005

Related U.S. Application Data
(60) Provisional application No. 60/639,083, filed on Dec. 22, 2004.

(51) Int. Cl. A61K 48/00; A61K 31/522; A61K 31/366; A61K 31/452; A61K 31/24

(52) U.S. Cl. 514/44; 514/263.32; 514/326; 514/456; 514/534

(57) ABSTRACT

Mammalian hair growth is reduced by topically applying a composition including a survivin inhibitor.

### CLAIM OF PRIORITY

This application claims priority under 35 U.S.C. § 119(e) to U.S. patent application Ser. No. 60/639,083 filed on Dec. 22, 2004, the entire contents of which are hereby incorporated by reference.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

Survivin has a role in controlling cell proliferation and suppression of apoptosis. The term “survivin”, as used herein, refers to the protein expressed by the survivin gene.

Survivin expression is highly regulated in cell cycle-dependent manner, and survivin is required for cytokinesis and chromosome movement during cell division. In addition, survivin inhibits cell death induced by various apoptotic stimuli. Over-expression of the survivin in vivo increases cell resistance to apoptosis. Transgenic expression of the survivin in epidermal keratinocytes has been shown to significantly reduce the number of apoptotic cells in the epidermis following exposure to UV irradiation. In contrast, the inhibition of expression of the survivin in vitro, by antisense survivin oligonucleotide treatment, increases the susceptibility of numerous cell lines to apoptosis. It has been suggested that survivin might suppress apoptosis by either directly or indirectly inhibiting the activity of caspases, the cell death proteases that induce apoptosis. In particular, the structural characteristics of survivin suggest an interaction between survivin and caspase-9. Loss of survivin phosphorylation results in the dissociation of an immunoprecipitable survivin-caspase-9 complex on the mitotic apparatus, allowing activation of caspase-9 dependent apoptosis. In addition, survivin may inhibit caspase activity indirectly by binding to a second mitochondrial-derived activator of caspases (Smac/DIABLO), thus preventing Smac/DIABLO execution of the mitochondrial apoptosis pathway. Survivin expression was seen in embryonic tissues as well as in the majority of human cancers, but its expression appears to be limited in normal adult tissues.

### SUMMARY

In one aspect, the invention provides a method (typically a cosmetic method) of reducing unwanted mammalian (preferably human) hair growth by applying to the skin a survivin inhibitor in an amount effective to reduce hair growth. The unwanted hair growth may be undesirable from a cosmetic standpoint or may result, for example, from a disease or an abnormal condition (e.g., hirsutism).

Survivin inhibitors include compounds that specifically inhibit the function of survivin protein in hair follicle by strongly interacting with the protein; compounds that reduce the levels of survivin in hair follicles, and compounds that inhibit activity of survivin, for example, by phosphorylation of survivin protein. “Strongly interacts” means the compound binds or preferentially binds survivin.

Typically, in practicing the aforementioned method, the inhibitor will be included in a topical composition along with a dermatologically or cosmetically acceptable vehicle. Accordingly, the present invention also relates to topical compositions comprising a dermatologically or cosmetically acceptable vehicle and a survivin inhibitor.

In addition, the present invention relates to the use of a survivin inhibitor for the manufacture of a therapeutic topical composition for reducing hair growth.

Specific compounds include both the compound itself and pharmacologically acceptable salts of the compound.

Other features and advantages of the invention may be apparent from the detailed description and from the claims.

### DETAILED DESCRIPTION

A preferred composition includes a survivin inhibitor in a cosmetically and/or dermatologically acceptable vehicle. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

Examples of some known survivin inhibitors are provided in Table 1.

TABLE 1

| Name of | | | |
|---|---|---|---|
| Inhibitor | Chemical Name(s) | Function | References |
| G₄N | meso-1,4-bis[(3,4- | Reduces survivin | Rhu Chih C. Huang |
| | dimethylaminoacetoxy)phenyl]- | levels | and |
| | (2R,3S)-dimethylbutane | | Jonathan D Heller, |
| | hydrochloride salt; | | US patent |
| | tetra-N,N-dimethylaminoglycine- | | application no. US |
| | NDGA ester; | | 2003/0171416 A1, |
| | | | 11 Sept. 2003 |
| M₄N | meso-1,4-bis(3,4- | Reduces survivin | Rhu Chih C. Huang |
| | dimethoxyphenyl)-(2R,3S)- | levels | and |
| | dimethylbutane; | | Jonathan D Heller, |
| | tetra-O-methyl-NDGA | | US patent |
| | | | application no. US |
| | | | 2003/0171416 A1, |
| | | | 11 Sept. 2003 |
| Roscovitine | (2R)-2-[[9-(1-methylethyl)-6- | Reduces survivin | Kim EH et al, |
| | [(phenylmethyl)amino]-9H-purin- | levels | Oncogene. 2004 |
| | 2-yl]amino]-1-nutanol | | Jan 15; 23(2): 446-56. |
| | (R)-2-[[9-(1-methylethyl)-6- | | |
| | [(phenylmethyl)amino]-9H-purin- | | |
| | 2-yl]amino]-1-butanol; | | |
| | CYC 202 | | |
| Silibinin | (2R,3R)-2-[(2R,3R)-2,3-dihydro- | Reduces survivin | Tyagi AK et al, |
| | 3-(4-hydroxy-3-methoxyphenyl)- | levels | Biochem Biophys |
| | 2-(hydroxymethyl)-1,4- | | Res Commun. |
| | benzodioxin-6-yl]-2,3-dihydro- | | 2003; 312(4): 1178-84. |
| | 3,5,7-trihydroxy-4H-1- | | |
| | benzopyran-4-one | | |
| | 3,5,7-trihydroxy-2-[3-(4-hydroxy- | | |
| | 3-methoxyphenyl)-2- | | |
| | (hydroxymethyl)-1,4- | | |
| | benzodioxan-6-yl]-4-chromanone; | | |
| | [2R-[2α,3β,6(2R*,3R*)]]-2-[2,3- | | |
| | dihydro-3-(4-hydroxy-3- | | |
| | methoxyphenyl)-2- | | |
| | (hydroxymethyl)-1,4-benzodioxin- | | |
| | 6-yl]-2,3-dihydro-3,5,7- | | |
| | trihydroxy-4H-1-benzopyran-4- | | |
| | one, | | |
| | Silybin; | | |
| | 1,4-benzodioxin, 4H-1- | | |
| | benzopyran-4-one deriv.; | | |
| | 7C3MT; | | |
| | Silliver; | | |
| | Silybin A; | | |
| | Silybin b1; | | |
| | Silybine; | | |
| | Silybum substance E6; | | |
| | Silymarin I; | | |
| | Silymarin MZ 80; | | |
| | Silymarine I | | |
| Resveratrol | 5-[(1E)-2-(4- | Reduces survivin | Hayashibara et al; |
| | hydroxyphenyl)ethenyl]-1,3- | levels | Nutr Cancer; 2002; |
| | benzenediol | | 44: 193-201 |
| | (E)-5-[2-(4- | | |
| | hydroxyphenyl)ethenyl]-1,3- | | |
| | benzenediol; | | |
| | 3,4′,5-Stilbenetriol; | | |
| | Resveratrol; | | |
| | (E)-5-(p-Hydroxystyryl)resorcinol; | | |
| | (E)-Resveratrol; | | |
| | 3,4′,5-Trihydroxy-trans-stilbene; | | |
| | CA 1201; trans-Resveratrol | | |
| Geranylgeranyl- | GGTI-298; | Survivin binding | Dan et al, |
| transferase I | GGTI-2166; | agent | Oncogene. 2004; |
| inhibitors | L-leucine, N-[4-[[(2R)-2-amino-3- | | 23(3): 706-15 |
| (GGTIs) | mercaptopropyl]amino]-2-(1- | | |
| | naphthalenyl)benzoyl]-, methyl | | |
| | ester (9CI); | | |
| | L-leucine, N-[4-[(2-amino-3- | | |
| | mercaptopropyl)amino]-2-(1- | | |
| | naphthalenyl)benzoyl]-, methyl | | |
| | ester, (R)- | | |
| Flavopiridol | 2-(2-chlorophenyl)-5,7-dihydroxy- | Inhibition of | Wall N et al., |
| | 8-[(3S,4R)-3-hydroxy-1-methyl-4- | survivin | Cancer Research |
| | piperidinyl]-4H-1-benzopyran-4- | phosphorylation | (2003), 63(1), 230-235. |
| | one | on Thr34 | |
| | cis-(−)-2-(2-chlorophenyl)-5,7- | | |
| | dihydroxy-8-(3-hydroxy-1-methyl- | | |
| | 4-piperidinyl)-4H-1-benzopyran- | | |
| | 4-one; | | |
| | Alvocidib | | |
| | HMR 1275; | | |
| | L 86-8275 | | |
| Survivin siRNA | | Silencing of | Ning S, et al., |
| | | survivin gene | Int J Oncol. 2004 |
| | | expression by | Oct; 25(4): 1065-71. |
| | | RNA interference | |
| Antisense | | Reducing survivin | U.S. Pat. No. 6165788: |
| molecules | | expression and | Antisense |
| targeted to | | therefore survivin | modulation of |
| survivin | | levels | survivin expression |

Synthesis of meso-1,4-bis[(3,4-dimethylaminoacetoxy)phenyl]-(2R,3S)-dimethylbutane hydrochloride salt (G₄N)

All four phenoxy groups of nordihydroguaiaretic acid (NDGA) were coupled to N,N-dimethylaminoglycine using the active ester method at 25° C. (Rhu Chih C. Huang and Jonathan D Heller, U.S. published application US 2003/0171416 A1, 11 Sep. 2003). The product (G₄N) was purified by chromatography using silica gel and characterized by LCMS. The protonated species was prepared in situ by the addition of dilute hydrochloric acid. G₄N has the following structure:

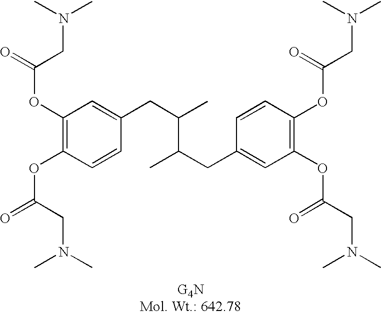

Specifically, to a solution of NDGA (0.342 g, 1.13 mmol) and N,N-dimethylglycine (0.7 g, 6.8 mmol ) in dichloromethane (7 ml) was added dicyclohexylcarbodiimide (DCC, 1.4 g, 6.8 mmol) and N,N-dimethylaminopyridine (DMAP, 62 mg, 0.5 mmol). The reaction mixture was allowed to stir for 24 hours under an argon atmosphere at 25° C. After filtration of the resultant dicyclohexylurea, the filtrate was washed with saturated aqueous sodium bicarbonate. The organic solvent was dried over anhydrous sodium sulfate, followed by evaporation to give the crude product mixture. The crude product residue was purified by chromatography on a preconditioned (chloroform) silica gel cartridge (BondElute® 10 g) using methanol-chloroform mixtures (1-5%) as eluant. Product containing fractions (test by thin layer chromatography) were combined and evaporated under reduced pressure to give pure G₄N (588 mg) in 81% yield as a viscous clear oil. Neutralization of 10% (w/w) solution in 5% methanol-water gave an in situ protonated product form of G₄N. LCMS (ESI+ve) m/z 665 (M⁺+1+Na) and 643 (M⁺+1).

The composition may include more than one survivin inhibitor. In addition, the composition may include one or more other types of hair growth reducing agents, such as those described in U.S. Pat. No. 4,885,289; U.S. Pat. No. 4,720,489; U.S. Pat. No. 5,132,293; U.S. Pat. No. 5,096,911; U.S. Pat. No. 5,095,007; U.S. Pat. No. 5,143,925; U.S. Pat. No. 5,328,686; U.S. Pat. No. 5,440,090; U.S. Pat. No. 5,364,885; U.S. Pat. No. 5,411,991; U.S. Pat. No. 5,648,394; U.S. Pat. No. 5,468,476; U.S. Pat. No. 5,475,763; U.S. Pat. No. 5,554,608; U.S. Pat. No. 5,674,477; U.S. Pat. No. 5,728,736; U.S. Pat. No. 5,652,273; WO 94/27586; WO 94/27563; and WO 98/03149, all of which are incorporated herein by reference.

The concentration of the survivin inhibitor in the composition may be varied over a wide range up to a saturated solution, preferably from 0.1% to 30% by weight or even more; the reduction of hair growth increases as the amount of inhibitor applied increases per unit area of skin. The maximum amount effectively applied is limited only by the rate at which the inhibitor penetrates the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, palmitic acid, oleic acid, and myristyl myristate. Solvents include ethyl alcohol, isopropanol, acetone, diethylene glycol, ethylene glycol, dimethyl sulfoxide, and dimethyl formamide.

The composition optionally can include components that enhance the penetration of the inhibitor into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the inhibitor. The composition also may be formulated to evaporate slowly from the skin, allowing the inhibitor extra time to penetrate the skin.

A cream-based topical composition containing a survivin inhibitor is prepared by mixing together water and all water soluble components in a mixing vessel-A. The pH is adjusted in a desired range from about 3.5 to 8.0. In order to achieve complete dissolution of ingredients the vessel temperature may be raised to up to 45° C. The selection of pH and temperature will depend on the stability of the survivin inhibitor. The oil soluble components, except for the preservative and fragrance components, are mixed together in another container (B) and heated to up to 70° C. to melt and mix the components. The heated contents of vessel B are poured into the water phase (container A) with brisk stirring. Mixing is continued for about 20 minutes. The preservative components are added at temperature of about 40° C. Stirring is continued until the temperature reaches about 25° C. to yield a soft cream with a viscosity of 8,000-12,000 cps, or a desired viscosity. The fragrance components are added at about 25° C.-30° C. while the contents are still being mixed and the viscosity has not yet built up to the desired range. If it is desired to increase the viscosity of the resulting emulsion, shear can be applied using a conventional homogenizer, for example a Silverson L4R homogenizer with a square hole high sheer screen. The topical composition can be fabricated by including the inhibitor in the water phase during the aforedescribed formulation preparation or can be added after the formulation (vehicle) preparation has been completed. The inhibitor can also be added during any step of the vehicle preparation. The components of the cream formulations are described in the examples below.

### EXAMPLE #1 (CREAM)

| INCI Name | w/w (%) |
|---|---|
| DI water | 61.00-75.00 |
| Survivin inhibitor^{a} | 1.00-15.00 |
| Mineral oil | 1.90 |
| Glyceryl stearate | 3.60 |
| PEG 100 Stearate | 3.48 |
| Cetearyl alcohol | 2.59 |
| Ceteareth-20 | 2.13 |
| Dimethicone, 100 ct | 0.48 |
| Lipidure PMB^{b} | 3.00 |
| Advanced Moisture Complex^{c} | 5.00 |
| Stearyl alcohol | 1.42 |
| Preservative, fragrance and color pigment | qs |
| Total | 100.00 |

^{a}An inhibitor can be selected, for example, from the list provided in Table 1.
^{b}Polyquartinium-51 (Collaborative Labs, NY).
^{c}Glycerin, water, sodium PCA, urea, trehalose, polyqauternium-51, and sodium hyaluronate (Collaborative Labs, NY).

### EXAMPLE #2 (CREAM)

| INCI Name | w/w (%) |
|---|---|
| Survivin inhibitor^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| Glyceryl triacetate (triacetin) | 0.5-10 |
| Preservative, fragrance and color pigment | q.s. |
| Water | q.s. to 100.00 |

^{a}An inhibitor can be selected, for example, from the list provided in Table 1.

### EXAMPLE #3 (CREAM)

| INCI Name | w/w (%) |
|---|---|
| Survivin inhibitor^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| 1-dodecyl-2-pyrrolidanone | 0.5-10% |
| Preservative, fragrance and color | q.s. |
| Water | to 100.00 |

^{a}An inhibitor can be selected, for example, from the list provided in Table 1.

### EXAMPLE #4 (CREAM)

| INCI Name | w/w (%) |
|---|---|
| Water | 70 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| 1-Dodecyl-2-pyrrolidanone | 1-10 |
| Total | 100.00 |

An inhibitor is added to the example 4 formulation and mixed until solubilized. An inhibitor can be selected, for example, from the list provided in Table 1.

### EXAMPLE #5 (CREAM)

| INCI Name | w/w (%) |
|---|---|
| Water | 70-80 |
| Glyceryl Stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Monocaprylate/caprate (Estol 3601, Uniquema, NJ) | 1-10 |
| Total | 100.00 |

An inhibitor is added to the example 5 formulation and mixed until solubilized. An inhibitor can be selected, for example, from the list provided in Table 1.

### EXAMPLE #6 (CREAM)

| INCI Name | w/w (%) |
|---|---|
| Water | 70-80 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| cis Fatty acids | 1-10 |
| Total | 100.00 |

An inhibitor is added to the example 6 formulation and mixed until solubilized. An inhibitor can be selected, for example, from the list provided in Table 1.

### EXAMPLE #7 (CREAM)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Terpene(s) | 1-10 |
| Total | 100.00 |

A survivin inhibitor is added to the example 7 formulation and mixed until solubilized. An inhibitor can be selected, for example, from the list provided in Table 1.

### EXAMPLE #8 (CREAM)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Polyoxyethylene sorbitans (tween) | 1-10 |
| Total | 100.00 |

A survivin inhibitor is added to the example 8 formulation and mixed until solubilized. An inhibitor can be selected, for example, from the list provided in Table-1.

A hydroalcoholic formulation containing a survivin inhibitor is prepared by mixing the formulation components in a mixing vessel. The pH of the formulation is adjusted to a desired value in the range of 3.5-8.0. The pH adjustment can also be made to cause complete dissolution of the formulation ingredients. In addition, heating can be applied to up to 45° C., or even up to 70° C. depending on the stability of the active in order to achieve dissolution of the formulation ingredients. Several hydroalcoholic formulations are listed below.

### EXAMPLE #9 (Hydro-alcoholic)

| INCI Name | w/w (%) |
|---|---|
| Water | 48.00-62.50 |
| Survivin inhibitor^{a} | 0.5-15.00 |
| Ethanol | 16.00 |
| Propylene glycol | 5.00 |
| Dipropylene glycol | 5.00 |
| Benzyl alcohol | 400 |
| Propylene carbonate | 2.00 |
| Captex-300^{b} | 5.00 |
| Total | 100.00 |

^{a}An inhibitor can be selected, for example, from the list provided in Table 1.
^{b}Caprylic/capric triglyceride (Abitec Corp., OH).

### EXAMPLE #10 (Hydro-alcoholic)

| INCI Name | w/w (%) |
|---|---|
| Water | 53.00-67.9 |
| Survivin inhibitor^{a} | 0.1-15.00 |
| Ethanol | 16.00 |
| Propylene glycol | 5.00 |
| Dipropylene glycol dimethyl ether | 5.00 |
| Benzyl alcohol | 4.00 |
| Propylene carbonate | 2.00 |
| Total | 100.00 |

^{a}An inhibitor can be selected, for example, from the list provided in Table 1.

### EXAMPLE #11 (Hydro-alcoholic)

| INCI Name | w/w (%) |
|---|---|
| Ethanol (alcohol) | 80 |
| Water | 17.5 |
| Propylene glycol dipelargonate | 2.0 |
| Propylene glycol | 0.5 |
| Total | 100.00 |

A survivin inhibitor is added to the formulation and mixed until solubilized. An inhibitor can be selected, for example, from the list provided in Table 1.

The composition should be applied topically to a selected area of the body from which it is desired to reduce hair growth. For example, the composition can be applied to the face, particularly to the beard area of the face, i.e., the cheek, neck, upper lip, and chin. The composition also may be used as an adjunct to, for example, shaving or mechanical epilation.

The composition can also be applied to the legs, arms, torso or armpits. The composition is particularly suitable for reducing the growth of unwanted hair in women having hirsutism or other conditions. In humans, the composition should be applied once or twice a day, or even more frequently, to achieve a perceived reduction in hair growth. Perception of reduced hair growth could occur as early as 24 hours or 48 hours (for instance, between normal shaving intervals) following use or could take up to, for example, three months. Reduced hair growth can be demonstrated quantitatively by reduced hair length, hair diameter, hair pigmentation, and/or hair density in the treated area. Reduced hair growth can be demonstrated cosmetically by less visible hair, shorter hair stubble, finer/thinner hair, softer hair, and/or a longer-lasting shave in the treated area.

Human Hair Follicle Growth Assay

Human skin was obtained from a plastic surgeon as a by-product of face-lift procedures. The skin samples generally consisted of haired and non-haired regions taken from the area of the face. Immediately after removal, the skin was placed in Williams E medium containing antibiotics, and kept refrigerated. The Williams E medium was commercially obtained (Life Technologies, Gaithersburg, Md.), and has been formulated with essential nutrients for maintaining viability of hair follicle in an in-vitro environment.

Human hair follicles in growth phase (anagen) were isolated from face-lift tissue under a dissecting scope using a scalpel and watchmakers forceps. The skin was sliced into thin strips exposing 2-3 rows of follicles that could readily be dissected. Follicles were placed into 0.5 ml Williams E medium supplemented with 2 mM L-glutamine, 10 ug/ml insulin, 10 ng/ml hydrocortisone, 100 units penicillin, 0.1 mg/ml streptomycin and 0.25 μg/ml amphotericin B. The follicles were incubated in 24 well plates (1 follicle/well) at 37° C. in an atmosphere of 5% CO₂ and 95% air. Hair follicle images were taken in the 24-well plates under the dissecting scope under a power of 20×. Hair follicle lengths were measured on day 0 (day follicles were placed in culture) and again on day 6-7. In this system follicles appear to fully differentiate into a hair fiber and increase in length at a rate similar to the human, in vivo, rate of about 0.3 mm/day. For testing survivin inhibitors, the inhibitor was included in the culture medium from time 0 and remained in the medium throughout the course of the experiment.

Immunohistochemistry Assay

Eight-micron cryosections through hair follicles or quick frozen skin biopsy were prepared and fixed in acetone for 10 minutes −20° C. For the immunodetection of survivin, the tyramide-amplification method was used. Briefly, after blocking of endogenous peroxidase and non-specific avidin/biotin binding (Avidin/Biotin blocking kit, Vector Lab), sections were incubated in TNB buffer (0.1 M Tris-HCl, pH 7.5, 0.15 M NaCl, and 0.5% Blocking Reagent, Perkin Elmer, Boston, Mass.) for 30 min. Then rabbit polyclonal antibody against human Survivin (Chemicon Int.; AB16532) was applied (1:1000, overnight), followed by application of the biotinylated goat anti-rabbit or goat anti-rabbit antiserum, diluted in TNB blocking buffer (Perkin Elmer, Boston, Mass., 1:200, 30 min). Subsequently, sections were incubated in streptavidin-horse radish peroxidase (1:100 in TNB, 30 min). Three washes with TNT buffer (0.1 M Tris-HCl, pH 7.6, 0.15M NaCl, 0.05% Tween) were followed by a 10 min application of TRITC-tyramide (1:50 in Amplification Diluent, Perkin Elmer, Boston, Mass.). ext, sections were counterstained with Hoechst 33342 (1:300, 15 min) for identification of cell nuclei, and mounted using VectaShield (Vector Laboratories).

For simultaneous detection of proliferating and survivin positive cells the protocol described above was combined with indirect immunofluorescence protocol using rabbit monoclonal antibody against Ki-67 (Dako; M 7187). After survivin detection, skin sections were incubated with antibody against Ki-67 overnight at room temperature, followed by incubation with TRITC-labeled goat ant-rabbit IgG (Jackson ImmunoResearch; 45 min, 37° C.). Counterstaining was performed using HOECHST 33342 dye (10 mg/ml in PBS; 10 minutes). Each phase was interspersed by a washing step (three times) in PBS. Finally, sections were mounted using VectaShield Vector Laboratories).

All sections were examined under a Olympus BX 60 fluorescent microscope and photodocumented with the help of a digital image analysis system (CoolSnap™ cooled CCD camera, Alpha Innotech).

Determination of Hair Follicle Survivin Protein Content by ELISA (Enzyme-Linked Immunosorbent Assay)

For protein extraction, 5-micron cryosections of the hair follicles were collected into the centrifuge tubes, followed by short time incubation with the lysis buffer (BD Bioscience; CA; Cat #K 1848-1). The solutions were centrifuged for 30 minutes at 14,000×g at 4° C., and the supernatants were frozen and stored at −80° C. For quantification of survivin protein, a commercially available ELISA kit was used, following the manufacturer's instructions (Assay Design Inc; Michigan; Cat # 900-111). Total protein concentration in the extracts was determined by using the bicinchoninic acid (BCA) protein assay (Pierce Chemical Company, Rockford; Cat #23225). The readings of optical density were performed, using EL340 Bio Kinetics microplate reader (Bio-Tek Instruments Inc). The survivin levels (in pg/ml) were normalized against the total amount of protein (in ug/ml) extracted from hair follicles.

Golden Syrian Hamster Assay

Male intact Golden Syrian hamsters are considered acceptable models for human beard hair growth in that they display oval shaped flank organs, one on each side, each about 8 mm. in major diameter. These organs produce fine light colored hair typical of the animal pelage found on the body. In response to androgens the flank organs produce dark coarse hair similar to male human beard hair. To evaluate the effectiveness of a composition, the flank organs of each of a group of hamsters are depilated by applying a thioglycolate-based chemical depilatory (Surgex) and/or shaved. To one organ of each animal 10 μl of vehicle alone once a day is applied, while to the other organ of each animal an equal amount of vehicle containing a survivin inhibitor. After thirteen applications (one application per day for five days a week), the flank organs are shaved and the amount of recovered hair (hair mass) from each is weighed. For certain experiments, where indicated, the treatment period was for less than 13 applications. The reduced treatment period allowed for determination of onset in activity. Percent-reduction of hair growth is calculated by subtracting the hair mass (mg) value of the test compound treated side from the hair mass value of the vehicle treated side; the delta value obtained is then divided by the hair mass value of the vehicle treated side, and the resultant number is multiplied by 100. Visual evaluations comparing hair regrowth between the drug treated and the vehicle control site were made generally on day-8, day-15 and on day-19. These observations provide an identification of onset in activity (and thus efficacy).

Results

Using immunohistochemical methodology, the presence of survivin was demonstrated in human scalp and beard-derived hair follicles hair follicles in vitro. In both cases survivin protein expression was restricted to the individual cells of the hair matrix and was seen in distinct cells along the outer root sheath. In beard hair follicles, a few survivin positive cells were also seen in the dermal papilla. Double immunostaining for simultaneous detection of survivin and proliferative marker Ki-67 revealed that only proliferating cells express survivin (see Figure).

A dose-dependent reduction of human hair follicle growth was seen with meso-1,4-bis[(3,4-dimethylaminoacetoxy)phenyl]-(2R,3S)-dimethylbutane hydrochloride salt (G₄N) (Table 2).

TABLE 2

| Compound | Dose | Length Increase (mm) | % Inhibition | p< |
|---|---|---|---|---|
| Control* | — | 1.7 ± 0.1 | 0 | |
| G4N | 20 uM | 1.19 ± 0.11 | 30 | 0.001 |
| G4N | 50 uM | 0.61 ± 0.14 | 64 | 0.000001 |

*Hair growth was determined by subtracting total hair follicle length on day 0 from total hair follicle length on day 6. % Inhibition = 100 − (hair growth for inhibitor treated follicles/hair growth for control) × 100.

Inhibition of flank organ hair growth in the Golden Syrian Hamster Assay was demonstrated following the topical administration of G4N (see Table 3), and silibinin and silymarin (see Table 4).

TABLE 3

| | | Treated Side | Vehicle Control Side | |
|---|---|---|---|---|
| Compound | Dose | (mg) | (mg)* | % Inhibition |
| G4N | 5% | 1.17 ± 0.2 | 2.21 ± 0.28 | 48 ± 6 |

*The vehicle was 20% propylene glycol in ethanol.

TABLE 4

| Compound | | Treated Side | Vehicle Control | |
|---|---|---|---|---|
| Name | Dose | (mg) | Side (mg)* | % Inhibition |
| Silibinin | 10% | 0.77 ± .14 | 2.07 ± .24 | 62.1 + 5.5 |
| Silymarin | 10% | 1.15 ± .14 | 2.51 ± .20 | 52.7 + 6.0 |

*The vehicle was 10% DMSO in ethanol, water, emerest, and propylene glycol.

Table 5 shows the dose-dependent reduction of human hair follicle growth in vitro by roscovitine.

TABLE 5

| Treatment | Length Increase in mm | Hair Growth Inhibition |
|---|---|---|
| Control | 1.7 + 0.2 | 42% |
| 10 uM Roscovitine | 0.97 + 0.13 | |
| Control | 1.4 ± 0.1 | 60% |
| 15 uM Roscovitine | 0.5 ± 0.1 | |
| Control | 1.2 ± 0.1 | 80% |
| 20 uM Roscovitine | 0.05 ± 0.05 | |

Table 6 shows the dose-dependent reduction in survivin protein levels in human hair follicles in vitro by roscovitine as determined by ELISA assay.

TABLE 6

| Treatment | Survivin Protein (pg/mg) | Inhibition |
|---|---|---|
| Control | 0.07 | 62% |
| 15 uM Roscovitine | 0.03 | |
| Control | 0.15 | 95% |
| 20 uM Roscovitine | 0.007 | |

Other embodiments are within the claims.

What is claimed is:

1. A method of reducing mammalian hair growth which comprises selecting an area of skin from which reduced hair growth is desired; and applying to said area of skin a dermatologically acceptable composition comprising a survivin inhibitor in an amount effective to reduce hair growth.

2. The method of claim 1, wherein said inhibitor is meso-1,4-bis[(3,4-dimethylaminoacetoxy)phenyl]-(2R,3S)-dimethylbutane hydrochloride salt.

3. The method of claim 1, wherein said inhibitor is meso-1,4-bis(3,4-dimethoxyphenyl)-(2R,3S)-dimethylbutane.

4. The method of claim 1, wherein said inhibitor is (2R)-2-[[9-(1-methylethyl)-6-[(phenylmethyl)amino]-9H-purin-2-yl]amino]-1-nutanol.

5. The method of claim 1, wherein said inhibitor is 2-(2-chlorophenyl)-5,7-dihydroxy-8-[(3S,4R)-3-hydroxy-1-methyl-4-piperidinyl]-4H-1-benzopyran-4-one.

6. The method of claim 1, wherein said inhibitor is (2R,3R)-2-[(2R,3R)-2,3-dihydro-3-(4-hydroxy-3-methoxyphenyl)-2-(hydroxymethyl)-1,4-benzodioxin-6-yl]-2,3-dihydro-3,5,7-trihydroxy-4H-1-benzopyran-4-one.

7. The method of claim 1, wherein said inhibitor is 5-[(1E)-2-(4-hydroxyphenyl)ethenyl]-1,3-benzenediol.

8. The method of claim 1, wherein said inhibitors inhibit geranylgeranyltransferase I.

9. The method of claim 1, wherein said inhibitor is L-leucine, N-[4-[[(2R)-2-amino-3-mercaptopropyl]amino]-2-(1-naphthalenyl)benzoyl]-, methyl ester (9CI).

10. The method of claim 1, wherein said inhibitor is L-leucine, N-[4-[(2-amino-3-mercaptopropyl)amino]-2-(1-naphthalenyl)benzoyl]-, methyl ester, (R)-.

11. The method of claim 1, wherein said inhibitor is 2-(2-chlorophenyl)-5,7-dihydroxy-8-[(3 S,4R)-3-hydroxy-1-methyl-4-piperidinyl]-4H-1-benzopyran-4-one.

12. The method of claim 1, wherein said inhibitor is an siRNA compound targeted mRNA of survivin.

13. The method of claim 1, wherein said inhibitor is an antisense compound targeted to nucleic acids encoding survivin.

14. The method of claim 1, wherein said inhibitor is a compound that strongly interacts with survivin in hair follicle cells.

15. The method of claim 1, wherein said inhibitor is a compound that reduces the level of survivin in hair follicles.

16. The method of claim 1, wherein said inhibitor is a compound that reduces expressions of survivin mRNA in hair follicles.

17. The method of claim 1, wherein said inhibitor is a compound that inhibits the phosphorylation of survivin

18. The method of claim 1, wherein the concentration of said inhibitor in said composition is between 0.1% and 30%.

19. The method of claim 1, wherein said inhibitor is applied to the skin in an amount of from 10 to 3000 micrograms of said inhibitor per square centimeter of skin.

20. The method of claim 1, wherein said area of skin is on the face of a human

21. The method of claim 1, wherein said area of skin is on a leg or arm, in an armpit, and/or on the torso of a human.

22. The method of claim 1, wherein said hair growth comprises androgen stimulated hair growth.
